SEQUENCE LISTING (11 sequences)

SEQ ID NO: 1 - protein, 10 aa, synthetic construct
GFSLTSYGVH

SEQ ID NO: 2 - protein, 16 aa, synthetic construct
VIYADGSTNYNPSLKS

SEQ ID NO: 3 - protein, 12 aa, synthetic construct
ARAYGNYWYIDV

SEQ ID NO: 4 - protein, 11 aa, synthetic construct
KSSESVSNDVA

SEQ ID NO: 5 - protein, 7 aa, synthetic construct
YAFHRFT

SEQ ID NO: 6 - protein, 9 aa, synthetic construct
HQAYSSPYT

SEQ ID NO: 7 - protein, 118 aa, synthetic construct
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSS

SEQ ID NO: 8 - protein, 107 aa, synthetic construct
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIK

SEQ ID NO: 9 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 10 - protein, 445 aa, synthetic construct
QVQLQESGPGLVKPSETLSLTCTVSGFSLTSYGVHWIRQPPGKGLEWIGVIYADGSTNYNPSLKSRVTISKDTSKNQVSLKLSSVTAADTAVYYCARAYGNYWYIDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGGPSVFLFPPKPKDTLMISRTPEVTCVVVAVSQEDPEVQFNWYVDGVEVHNAKTKPREEQFNSTYRVVSVLTVVHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSKLTVDKSRWQEGNVFSCSVMHEALHNHYTQKSLSLSLGK

SEQ ID NO: 11 - protein, 214 aa, synthetic construct
DIVMTQSPDSLAVSLGERATINCKSSESVSNDVAWYQQKPGQPPKLLINYAFHRFTGVPDRFSGSGYGTDFTLTISSLQAEDVAVYYCHQAYSSPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0167667 A1
(43) Pub. Date: Jun. 18, 2026

(54) PREPARATION METHODS FOR A HIGHLY CONCENTRATED PD1 ANTIBODY SOLUTION BY APPLYING SINGLE-PASS TANGENTIAL FLOW FILTRATION (SPTFF)

(71) Applicant: BeOne Medicines I GmbH, Basel (CH)

(72) Inventors: Yu JI, Suzhou (CN); Xuening LI, Suzhou (CN); Dawei ZHOU, Suzhou (CN)

(73) Assignee: BeOne Medicines I GmbH, Basel (CH)

(21) Appl. No.: 19/294,029

(22) Filed: Aug. 7, 2025

Related U.S. Application Data
(63) Continuation of application No. PCT/CN2024/076654, filed on Feb. 7, 2024.

(30) Foreign Application Priority Data
Feb. 8, 2023 (WO) PCT/CN2023/074866

(51) Int. Cl. C07K 1/34; C07K 16/28

(52) U.S. Cl. CPC C07K 1/34; C07K 16/2818

(57) ABSTRACT

The present disclosure provides for a preparation method of highly concentrated antibody solution that binds to human programmed death receptor 1 (PD1). This process is able to manufacture the high concentrated antibody solution by an ultrafiltration/diafiltration (UF/DF) unit operation described herein. The UF/DF preparation method comprises a single-pass tangential flow filtration (SPTFF) as the first ultrafiltration concentration step, a buffer solution diafiltration step and a second ultrafiltration concentration step using conventional UF/DF. The process has a broad operation parameter range and maintains antibody stability and integrity.

### CROSS-REFERENCE TO RELATED APPLICATIONS

This application is a continuation of International Patent Application No. PCT/CN2024/076654, filed Feb. 7, 2024, which claims priority from International Patent Application No. PCT/CN2023/074866, filed Feb. 8, 2023, the contents of these applications are incorporated by reference in their entirety.

### SEQUENCE LISTING

The present application contains a sequence listing which has been submitted electronically in xml format, and is hereby incorporated by reference in its entirety. Said xml file was created on Feb. 7, 2024, is 11,510 bytes in size, and named 138881_1355_Sequence_Listing.xml.

### FIELD OF THE DISCLOSURE

The present disclosure is directed to methods of preparing a highly concentrated solution comprising antibodies or antigen binding fragments thereof that bind to human programmed death receptor 1 (PD1). This methodology is intended to manufacture a high concentrated antibody solution by applying single-pass tangential flow filtration (SPTFF) as described herein. High concentration antibody solutions are useful, for example, in subcutaneous administration. The SPTFF preparation method comprises a first ultrafiltration concentration step by SPTFF, a buffer solution diafiltration step and a second ultrafiltration concentration step by conventional tangential flow filtration. This process has a broad operating parameter range and demonstrates that the high concentration solution prepared by this method preserves the antibody quality characteristics through whole unit operation.

### BACKGROUND OF THE DISCLOSURE

With the rapid development of antibody therapeutics, more and more are turning to subcutaneous formulations as opposed to intravenous (IV) formulation and administration, in order to reduce the clinical cost and improve the compliance of patients. For the subcutaneous route of administration of monoclonal antibody injections, the dose administered is usually in the range of 50 mg to 800 mg, while the maximum subcutaneous volume is generally limited to about 2 ml, which provides for a nominal volume to be delivered in a short period of time. Therefore, highly concentrated protein preparations require additional processes to obtain protein concentrations of up to 100 mg/ml or more without detriment to the antibody itself.

In the manufacturing process, ultrafiltration/diafiltration (UF/DF) is typically the final process to obtain the antibody concentration in the range of 10-60 mg/ml. However, the antibody dose for intravenous infusion is about one hundred milligrams to about one gram. In order to achieve the same pharmacokinetics and efficacy in subcutaneous administration by injecting an antibody solution under the skin, the ideal target antibody concentration during UF/DF can be as high as 150 mg/m or above.

This high concentration creates technical challenges in the manufacturing process. First, highly concentrated antibody solutions can have high viscosity, which shows different hydrodynamic behavior in UF/DF. The mass transfer can be limited due to higher pressure on the membrane, resulting in decreased flux through the membrane, and can lead to membrane fouling. Secondly, there is a great difference between the initial feed protein concentration and the protein concentration in the final solution, during which 40 times concentration can be required. The volume change is also quite large, especially in commercial scale manufacturing. These factors play a role in the design of the UF/DF process and selection of skid. The UF/DF process setup should be able to handle large volume solution under high flowrate, and then be able to handle extreme low volume (10 or 20 times less) under relatively low flowrate for highly concentrated solution in the later processing phase. The range of pump and sensors, tubing diameter, flowmeter and dead volume cannot meet both the process requirements in the early phase and the later phase by using only one set of conventional UF/DF skid. The present disclosure provides for a novel preparation method of manufacturing a highly concentrated antibody solution by applying SPTFF in the UF/DF steps.

### SUMMARY OF THE INVENTION

The present disclosure provides a preparation method of a highly concentrated anti-human PD1 monoclonal antibody solution for subcutaneous administration by applying SPTFF unit operation in UF/DF process, where preferably the PD1 antibody is Tislelizumab. The process comprises the steps of.
1. Loading the feed material plus buffering means into the SPTFF system with a starting protein concentration after viral filtration;
2. Ultrafiltrating the solution to obtain UF1 pool with an intermediate concentration in the UF1 step by SPTFF;
3. Switching to another set of conventional tangential flow filtration skid, and diafiltrating the UF1 pool with DF buffer into the final drug substance formulation buffer, preferably His-His HCl buffer, to obtain the DF pool;
4. Ultrafiltrating the DF pool into high concentration protein solution as over concentrated pool with required concentration in the same skid as step 3;
5. Preparing the UF2 pool by combining the over concentrated pool with or without system flush, and diluting to the final high concentration formulation solution by adding surfactant, sugar stock solution to achieve the final drug substance target concentration.

In some embodiments, the feed material is in 50 mM acetate buffer, with different initial concentration from 8 g/L to 20 g/L. In some embodiments, the UF/DF membrane is Pellion3 Ultracel™ 30 kDa, D membrane in SPTFF format and in conventional membrane format. The membrane area can be adjusted according to the total protein amount for processing. In some embodiments, the SPTFF membrane area in UF1 is 0.33 m² and loading capacity is about 268.71 g/i. The conventional UF/DF membrane area in DF/UF2 is 0.11 m² and loading capacity is 806.14 g/m².

In some embodiments, the post-membrane pressure for UF1 SPTFF is in 10-20 Psi range, preferably ˜15 Psi. The feed flowrate can be 0.05-0.4 L/min/m² in UF1 SPTFF. The protein concentration of the UF1 pool after the first ultrafiltration step can be 30-70 g/L. In some embodiments, the VCF is from 2 to 10, preferably ˜5 in UF1 step.

In some embodiments, the transmembrane pressure (TMP) for DF is 14.5 Psi. The feed flux is 5 L/min/m² in DF. The starting protein concentration of UF1 pool for DF step is within 30-70 g/L, preferably 50 g/L. The exchange volume number in DF step should be larger than 4, preferably 6 or more.

In some embodiments, the TMP for UF2 is 14.5 Psi. The feed flux is 5 L/min/m². The feed flowrate can be adjusted by keeping the TMP relatively constant at target pressure. The adjustment can be processed manually or automatically through the Proportional-Integral-Derivative (PID) setting.

In some embodiments, the over concentrated pool in UF2 step can have concentration at any value from 50 g/L to 221 g/L at room temperature with a solution viscosity up to 300 mPa·s. The UF2 pool made from the over concentrated pool can have a required concentration from 50 g/L to 221 g/L at room temperature by diluting with DF buffer. The protein concentration of the UF2 pool for subcutaneous administration purposes requires ahigh concentration, preferably an antibody concentration higher than 150 g/L.

In some embodiments, the SPTFF UF1 pool, DF pool and the over concentrated pool are stable at room temperature for 1 hour and up to 5 hours. The quality data (SEC. CE-SDS(NR) and IEC) of protein are consistent during UF/DF process from UF1 to the over concentrated pool and a stable hold up to 5 hours at room temperature or at 35° C.

In some embodiments, the final high concentrated drug substance manufactured by the UF/DF process for subcutaneous administration according to the present disclosure has comparable quality data (SEC, CE-SDS(NR) and TEC) to the drug substance for intravenous infusion administration.

In some embodiments, the UF/DF unit operation is processed at 35° C. with buffers and all intermediate product pools kept at 35° C. The processing time at 35° C. can be ˜⅓ less than time at room temperature. The over concentrated pool and UF2 pool are able to achieve up to 230 g/L at 35° C.

In some embodiments, the viscosity of protein solution is about 3.0 mPa·s in SPTFF UF1 pool and DF pool, up to 258.8 mPa·s in over concentrated pool and UF2 pool. The UF/DF process and system can handle solutions in a broad range of viscosity, up to 300 mPa·s.

In some embodiments, the formulation buffer is selected from the group consisting of histidine, acetate, mixture of histidine and acetic acid. In some embodiments, the formulation buffer can be histidine buffer. In some embodiments, the concentration of histidine buffer is from about 10 mM to about 30 mM. In some embodiments, the concentration of the histidine buffer is about 20 mM histidine.

TABLE 1

| | | | pH Shift During | |
|---|---|---|---|---|
| | | | Freezing | |
| Acid for | Buffering | | pH at | Δ pH |
| Buffer | Range | pKa | 25° C. | at −20° C. |
| Phosphoric acid | Neutral-Basic | 2.1, 7.2, 12.3 | 7.2 | −1.8 |
| Citric acid | Acidic-Neutral | 3.1, 4.8, 6.4 | 6.2 | −0.2 |
| Acetic acid | Acidic | 4.8 | 5.6 | +0.5 |
| Histidine | Neutral | 1.8, 6.1, 9.2 | 5.4 | +0.8 |
| Lactic acid | Acidic | 3.9 | N/A | N/A |
| Tromethamine | Neutral-Basic | 8.1 | 7.2 | +2.1 |
| Gluconic acid | Acidic | 3.6 | N/A | N/A |
| Aspartic acid | Acidic | 2.1, 3.9, 9.8 | N/A | N/A |
| Glutamic acid | Acidic | 2.1, 4.1, 9.5 | N/A | N/A |
| Tartaric acid | Acidic | 3.2, 4.9 | 5.0 | −0.3 |
| Succinic acid | Acidic-Neutral | 4.2, 5.6 | 5.6 | +0.3 |
| Malic acid | Acidic-Neutral | 3.4, 5.1 | 5.0 | −0.3 |
| Fumaric acid | Acidic | 3.0, 4.4 | N/A | N/A |
| α-Ketoglutaric | Acidic-Neutral | 2.5, 4.7 | N/A | N/A |

N/A—Data not available

In some embodiments the PD1 antibody is tislelizumab (BGB-A317, Table 2) or an antigen binding fragment of tislelizumab. In some embodiments, the subcutaneous antibody formulation has an antibody concentration between about 50 mg to 800 mg. In another embodiment the subcutaneous antibody formulation has an antibody concentration of about 100 mg, about 150 mg, about 200 mg, about 250 mg, about 300 mg, about 350 mg, about 400 mg, about 450 mg, about 500 mg, about 550 mg or about 600 mg.

### BRIEF DESCRIPTION OF THE DRAWINGS

FIG. 1A. The process flowchart of high concentration UF/DF unit operation in detailed steps with SPTFF UF/DF/UF2 as main operation steps.

FIG. 1B. A diagram of SPTFF UF/DF/UF2 system designed for high concentration antibody solution processing.

FIG. 1C. Impacts of pressure and feed flux on VCF in SPTFF UF1 process.

FIG. 2. Impacts of feed flux and concentration on VCF in SPTFF UF1 process.

FIG. 3. Impacts of temperature on VCF in SPTFF UF1 process.

FIG. 4A. The process chart of SPTFF UF1 step with feed flux, protein concentration curves at post-membrane pressure 15 Psi at room temperature (RT).

FIG. 4B. The process chart of DF and UF2 steps with feed flux, protein concentration and permeate flux curves at TMP 14.5 Psi at room temperature.

FIG. 4C. The process chart of unit operation with protein concentration, osmolality and viscosity curves in SPTFF UF1/DF/UF2 steps at room temperature.

FIG. 4D. The quality data (SEC) comparison between feed load, SPTFF pool, DF pool and over concentrated pool in process operation at room temperature and hold up to 5 hours.

FIG. 4E. The quality data (CE-SDS NR) comparison between feed load, SPTFF pool, DF pool and over concentrated pool in the process operation at room temperature and hold up to 5 hours.

FIG. 4F. The quality data (IEC) comparison between feed load, SPTFF pool, DF pool and over concentrated pool in process operation at room temperature and hold up to 5 hours.

FIG. 5A. The process chart of SPTFF UF1 step with feed flux, protein concentration curves at post-membrane pressure 15 Psi and 35° C.

FIG. 5B. The process chart of DF and UF2 steps with feed flux, protein concentration and permeate flux curves at TMP 14.5 Psi and 35° C.

FIG. 5C. The process chart of unit operation with protein concentration, osmolality and viscosity curves in the SPTFF UFJ/DF/UF2 steps at 35° C.

FIG. 5D. The quality data (SEC) comparison between the feed load, SPTFF pool, DF pool and the over concentrated pool in process operation at 35° C. and hold up to 5 hours.

FIG. 5E. The quality data (CE-SDS NR) comparison between the feed load, SPTFF pool, DF pool and over concentrated pool in process operation at 35° C. and hold up to 5 hours.

FIG. 5F. The quality data (IEC) comparison between the feed load, SPTFF pool, DF pool and over concentrated pool in process operation at 35° C. and hold up to 5 hours.

### DETAILED DESCRIPTION OF THE DISCLOSURE

### Definitions

Unless specifically defined elsewhere in this document, all other technical and scientific terms used herein have the meaning commonly understood by one of ordinary skill in the art.

As used herein, including the appended claims, the singular forms of words such as ‘a,’ ‘an,’ and ‘the,’ include their corresponding plural references unless the context clearly dictates otherwise.

The term ‘or’ is used to mean, and is used interchangeably with, the term ‘and/or’ unless the context clearly dictates otherwise.

Throughout this specification and the claims which follow, unless the context requires otherwise, the word ‘comprise’ and variations such as ‘comprises’ and ‘comprising,’ will be understood to imply the inclusion of a stated amino acid sequence, DNA sequence, step or group thereof, but not the exclusion of any other amino acid sequence, DNA sequence, step. When used herein the term ‘comprising’ can be substituted with the term ‘containing’, ‘including’ or sometimes ‘having’.

The term ‘antibody’ herein is used in the broadest sense and specifically covers antibodies (including full length monoclonal antibodies) and antibody fragments so long as they recognize antigen, e.g., PD1. An antibody is usually monospecific, but may also be described as idiospecific, heterospecific, or polyspecific. Antibody molecules bind by means of specific binding sites to specific antigenic determinants or epitopes on antigens.

The term ‘monoclonal antibody’ or ‘mAb’ or ‘Mab’ herein means a population of substantially homogeneous antibodies, i.e., the antibody molecules comprised in the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their complementarity determining regions (CDRs), which are often specific for different epitopes. The modifier ‘monoclonal’ indicates the character of the antibody as being obtained from a substantially homogeneous population of antibodies and is not to be construed as requiring production of the antibody by any particular method. Monoclonal antibodies (mAbs) may be obtained by methods known to those skilled in the art. See, for example Kohler G et al., Nature 1975 256:495-497; U.S. Pat. No. 4,376,110; Ausubel F M et al., CURRENT PROTOCOLS IN MOLECULAR BIOLOGY 1992; Harlow E et al., ANTIBODIES: A LABORATORY MANUAL, Cold spring Harbor Laboratory 1988; and Colligan J E et al., CURRENT PROTOCOLS IN IMMUNOLOGY 1993. The mAbs disclosed herein may be of any immunoglobulin class including IgG, IgM, IgD, IgE, IgA, and any subclass thereof. A hybridoma producing a mAb may be cultivated in vitro or in vivo. High titers of mAbs can be obtained by in vivo production where cells from the individual hybridomas are injected intraperitoneally into mice, such as pristine-primed Balb/c mice to produce ascites fluid containing high concentrations of the desired mAbs. MAbs of isotype IgM or IgG may be purified from such ascites fluids, or from culture supernatants, using column chromatography methods well known to those of skill in the art.

In general, the basic antibody structural unit comprises a tetramer. Each tetramer includes two identical pairs of polypeptide chains, each pair having one ‘light chain’ (about 25 kDa) and one ‘heavy chain’ (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as α, δ, ε, γ, or μ, and define the antibody's isotypes as IgA, IgD, IgE, IgG, and IgM, respectively. Within light and heavy chains, the variable and constant regions are joined by a ‘J’ region of about 12 or more amino acids, with the heavy chain also including a ‘D’ region of about 10 more amino acids.

The variable regions of each light/heavy chain (VL/VH) pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called ‘complementarity determining regions (CDRs)’, which are located between relatively conserved framework regions (FR). The CDRs are usually aligned by the framework regions, enabling binding to a specific epitope. In general, from N-terminal to C-terminal, both light and heavy chain variable domains sequentially comprise FR-1 (or FR1), CDR-1 (or CDR1), FR-2 (FR2), CDR-2 (CDR2), FR-3 (or FR3), CDR-3 (CDR3), and FR-4 (or FR4). The assignment of amino acids to each domain is, generally, in accordance with the definitions of Sequences of Proteins of Immunological Interest, Kabat, et al., National Institutes of Health, Bethesda, Md.; 5th ed.; NIH Publ. No. 91-3242 (1991); Kabat (1978) Adv. Prot. Chem. 32: 1-75; Kabat, et al., (1977) J. Biol. Chem. 252:6609-6616; Chothia, et al, (1987) J Mol. Biol. 196:901-917 or Chothia, et al., (1989) Nature 342:878-883.

The term ‘hypervariable region’ means the amino acid residues of an antibody that are responsible for antigen-binding. The hypervariable region comprises amino acid residues from a ‘CDR’ (i.e., VL-CDR1, VL-CDR2 and VL-CDR3 in the light chain variable domain and VH-CDR1, VH-CDR2 and VH-CDR3 in the heavy chain variable domain). See, Kabat et al. (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure). The term ‘framework’ or ‘FR’ residues mean those variable domain residues other than the hypervariable region residues defined herein as CDR residues.

Unless otherwise indicated, ‘antibody fragment’ or ‘antigen-binding fragment’ means antigen binding fragments of antibodies, i.e., antibody fragments that retain the ability to bind specifically to the antigen bound by the full-length antibody, e.g., fragments that retain one or more CDR regions. Examples of antigen binding fragments include, but not limited to, Fab, Fab′, F(ab′)2, and Fv fragments; diabodies; linear antibodies; single-chain antibody molecules, e.g., single chain Fv (ScFv); nanobodies and multispecific antibodies formed from antibody fragments.

An antibody that binds to a specified target protein with specificity is also described as specifically binding to a specified target protein. This means the antibody exhibits preferential binding to that target as compared to other proteins, but this specificity does not require absolute binding specificity. An antibody is considered ‘specific’ for its intended target if its binding is determinative of the presence of the target protein in a sample, e.g., without producing undesired results such as false positives. Antibodies or binding fragments thereof, useful in the present disclosure will bind to the target protein with an affinity that is at least two-fold greater, preferably at least 10-times greater, more preferably at least 20-times greater, and most preferably at least 100-times greater than the affinity with non-target proteins. An antibody herein is said to bind specifically to a polypeptide comprising a given amino acid sequence.

The term ‘human antibody’ herein means an antibody that comprises human immunoglobulin protein sequences only. A human antibody may contain murine carbohydrate chains if produced in a mouse, in a mouse cell, or in a hybridoma derived from a mouse cell. Similarly, ‘mouse antibody’ or ‘rat antibody’ means an antibody that comprises only mouse or rat immunoglobulin protein sequences, respectively.

The term ‘humanized antibody’ means forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix ‘hum,’ ‘hu,’ ‘Hu’ or ‘h’ is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

Further, the antibody of the present application has potential therapeutic uses in controlling viral infections and other human diseases that are mechanistically involved in immune tolerance or ‘exhaustion’. In the context of the present application, the term ‘exhaustion’ refers to a process which leads to a depleted ability of immune cells to respond to a cancer or a chronic viral infection.

The term ‘trans-membrane pressure’ or ‘TMP’ is the pressure exerted on the UF/DF membrane. TMP is calculated by Equation (1) below:

$$TMP = \frac{{P}_{f e e d} + {P}_{retentate}}{2} - {P}_{permeate} \qquad Equation ( 1 )$$

in which, P_{reed}, Pᵣₑₜₑₙₜₐₜₑ, Pₚₑᵣₘₑₐₜₑ are pressure of feed inlet, retentate outlet and permeate outlet respectively.

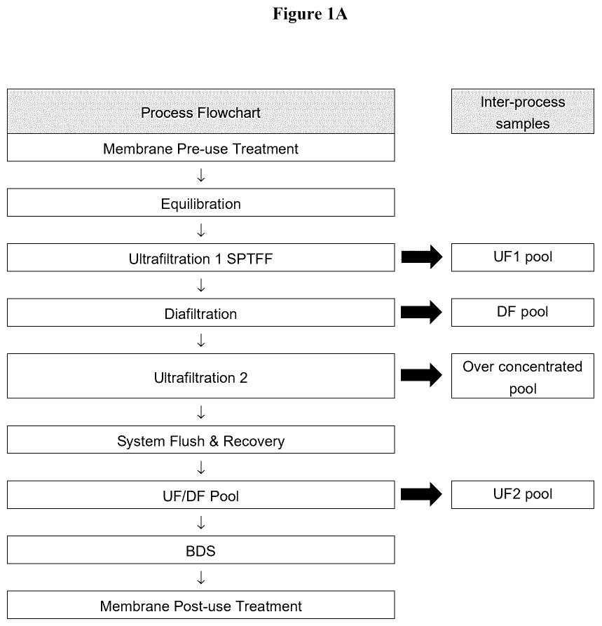

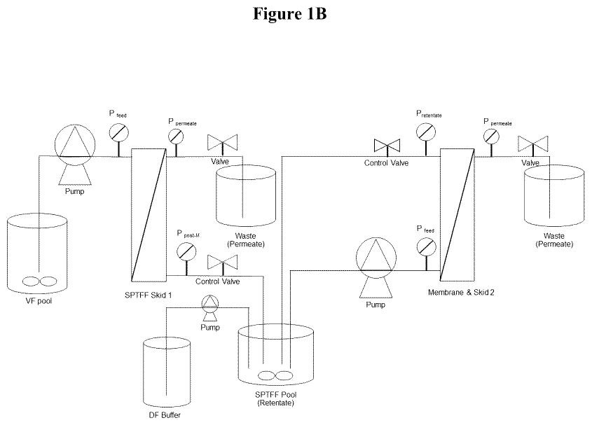

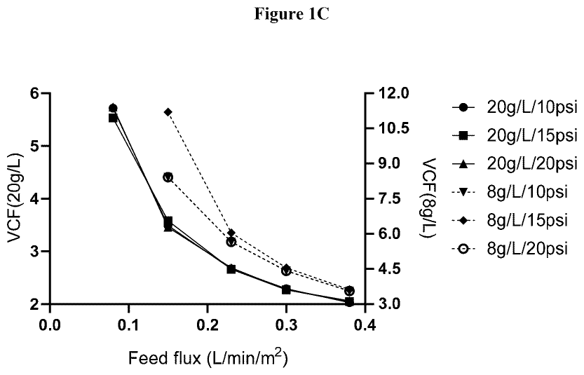

‘Ultrafiltration step 1’ (UF1) means the first ultrafiltration step in the process, this is shown in FIG. 1A. This step uses SPTFF according to the present disclosure.

‘Diafiltration step’ (DF) refers to any diafiltration step in the process, shown in FIG. 1A.

The term ‘ultrafiltration step 2’ (UF2) means ultrafiltration step 2 in the process, shown in FIG. 1A.

The abbreviation ‘VCF’ means ‘volume concentration factor,’ which is the amount that the feed stream has been reduced in volume from the initial volume calculated by Equation (2):

$$Equation ( 2 )$$

$$VCF = \frac{Total initial feed volume}{Current retentate volume} = \frac{Current concentration in retentate}{Initial concentration in feed}$$

The term ‘WFI’ means water for injection.

‘CIP’ means ‘clean-in-place.’

The term ‘NWP’ is an abbreviation of ‘normalized water permeability’. The NWP test is a method to assess the effectiveness of the membrane CIP process.

The term ‘permeate flux’ is defined as the solution flux through the IUF/DF membrane.

The term ‘RT’ is an abbreviation of ‘Room temperature’. It sets a temperature target at 20′C and within the range of 18-26′C.

### Anti-PD1 Antibody

The present disclosure provides for anti-PD1 antibodies and subcutaneous formulations thereof. For example, Tislelizumab (BGB-A317), is an anti-PD1 antibody disclosed in U.S. Pat. No. 8,735,553 with the sequences provided below.

TABLE 2
Tislelizumab sequences

| Domain | SEQ ID NO: | Amino Acid Sequence |
|---|---|---|
| HCDR1 | SEQ ID NO: 1 | GFSLTSYGVH |
| HCDR1 | SEQ ID NO: 2 | VIYADGSTNYNPSLKS |
| HCDR3 | SEQ ID NO: 3 | ARAYGNYWYIDV |
| LCDR1 | SEQ ID NO: 4 | KSSESVSNDVA |
| LCDR2 | SEQ ID NO: 5 | YAFHRFT |
| LCDR3 | SEQ ID NO: 6 | HQAYSSPYT |
| VH | SEQ ID NO: 7 | QVQLQESGPGLVKPSETLSLTCTVSGFSLTSYGVHWIRQPPGKGLE |
| | | WIGVIYADGSTNYNPSLKSRVTISKDTSKNQVSLKLSSVTAADTAV |
| | | YYCARAYGNYWYIDVWGQGTTVTVSS |
| VL | SEQ ID NO: 8 | DIVMTQSPDSLAVSLGERATINCKSSESVSNDVAWYQQKPGQPPK |
| | | LLINYAFHRFTGVPDRFSGSGYGTDFTLTISSLQAEDVAVYYCHQA |
| | | YSSPYTFGQGTKLEIK |
| IgG4 | SEQ ID NO: 9 | ASTKGPSVFPLAPCSRSTSESTAALGCLVKDYFPEPVTVSWNSGAL |
| constant | | TSGVHTFPAVLQSSGLYSLSSVVTVPSSSLGTKTYTCNVDHKPSNT |
| domain | | KVDKRVESKYGPPCPPCPAPPVAGGPSVFLFPPKPKDTLMISRTPE |
| | | VTCVVVAVSQEDPEVQFNWYVDGVEVHNAKTKPREEQFNSTYRV |
| | | VSVLTVVHQDWLNGKEYKCKVSNKGLPSSIEKTISKAKGQPREPQ |
| | | VYTLPPSQEEMTKNQVSLTCLVKGFYPSDIAVEWESNGQPENNYK |
| | | TTPPVLDSDGSFFLYSKLTVDKSRWQEGNVFSCSVMHEALHNHYT |
| | | QKSLSLSLGK |
| Heavy Chain | SEQ ID NO: 10 | QVQLQESGPGLVKPSETLSLTCTVSGFSLTSYGVHWIRQPPGKGLE |
| | | WIGVIYADGSTNYNPSLKSRVTISKDTSKNQVSLKLSSVTAADTAV |
| | | YYCARAYGNYWYIDVWGQGTTVTVSSASTKGPSVFPLAPCSRSTS |
| | | ESTAALGCLVKDYFPEPVTVSWNSGALTSGVHTFPAVLQSSGLYS |
| | | LSSVVTVPSSSLGTKTYTCNVDHKPSNTKVDKRVESKYGPPCPPCP |
| | | APPVAGGPSVFLFPPKPKDTLMISRTPEVTCVVVAVSQEDPEVQFN |
| | | WYVDGVEVHNAKTKPREEQFNSTYRVVSVLTVVHQDWLNGKEY |
| | | KCKVSNKGLPSSIEKTISKAKGQPREPQVYTLPPSQEEMTKNQVSL |
| | | TCLVKGFYPSDIAVEWESNGQPENNYKTTPPVLDSDGSFFLYSKLT |
| | | VDKSRWQEGNVFSCSVMHEALHNHYTQKSLSLSLGK |
| Light chain | SEQ ID NO: 11 | DIVMTQSPDSLAVSLGERATINCKSSESVSNDVAWYQQKPGQPPK |
| | | LLINYAFHRFTGVPDRFSGSGYGTDFTLTISSLQAEDVAVYYCHQA |
| | | YSSPYTFGQGTKLEIKRTVAAPSVFIFPPSDEQLKSGTASVVCLLNN |
| | | FYPREAKVQWKVDNALQSGNSQESVTEQDSKDSTYSLSSTLTLSK |
| | | ADYEKHKVYACEVTHQGLSSPVTKSFNRGEC |

Anti-PD1 antibodies can include, without limitation, Tislelizumab, Pembrolizumab or Nivolumab. Pembrolizumab (formerly MK-3475), as disclosed by Merck, in U.S. Pat. Nos. 8,354,509 and 8,900,587 is a humanized lgG4-K immunoglobulin which targets the PD1 receptor and inhibits binding of the PD1 receptor ligands PD-L1 and PD-L2. Pembrolizumab has been approved for the indications of metastatic melanoma and metastatic non-small cell lung cancer (NSCLC) and is under clinical investigation for the treatment of head and neck squamous cell carcinoma (HNSCC), and refractory Hodgkin's lymphoma (cHL). Nivolumab (as disclosed by Bristol-Meyers Squibb) is a fully human lgG4-K monoclonal antibody. Nivolumab (clone 5C4) is disclosed in U.S. Pat. No. 8,008,449 and WO 2006/121168. Nivolumab is approved for the treatment of melanoma, lung cancer, kidney cancer, and Hodgkin's lymphoma.

### Antibody Production

Anti-PD1 antibodies and antigen-binding fragments thereof can be produced by any means known in the art, including but not limited to, recombinant expression, chemical synthesis, and enzymatic digestion of antibody tetramers, whereas full-length monoclonal antibodies can be obtained by, e.g., hybridoma or recombinant production. Recombinant expression can be from any appropriate host cells known in the art, for example, mammalian host cells, bacterial host cells, yeast host cells, insect host cells, etc.

The disclosure further provides polynucleotides encoding the antibodies described herein, e.g., polynucleotides encoding heavy or light chain variable regions or segments comprising the complementarity determining regions as described herein. In some aspects, the polynucleotide encoding the heavy chain variable regions has at least 85%, 89%, 90%, 91%, 92%, 93%, 94%, 95%, 96%, 97%, 98%, 99%, or 100% nucleic acid sequence identity with a polynucleotide that encodes for the polypeptide of SEQ ID NO:7. In some aspects, the polynucleotide encoding the light chain variable regions has at least 85%, 89%, 90%, 91%, 92%, 93%, 94%, 95%, 96%, 97%, 98%, 99%, or 100% nucleic acid sequence identity with a polynucleotide that encodes for the poly peptide of SEQ ID NO:8.

The polynucleotides of the present disclosure can encode the variable region sequence of an anti-PD1 antibody. They can also encode both a variable region and a constant region of the antibody. Some of the polynucleotide sequences encode a polypeptide that comprises variable regions of both the heavy chain and the light chain of one of the exemplified Tislelizumab antibodies.

Also provided in the present disclosure are expression vectors and host cells for producing the Tislelizumab antibodies. The choice of expression vector depends on the intended host cells in which the vector is to be expressed. Typically, the expression vectors contain a promoter and other regulatory sequences (e.g., enhancers) that are operably linked to the polynucleotides encoding a Tislelizumab antibody chain or antigen-binding fragment. In some aspects, an inducible promoter is employed to prevent expression of inserted sequences except under the control of inducing conditions. Inducible promoters include, e.g., arabinose, lacZ, metallothionein promoter or a heat shock promoter. Cultures of transformed organisms can be expanded under non-inducing conditions without biasing the population for coding sequences whose expression products are better tolerated by the host cells. In addition to promoters, other regulatory elements can also be required or desired for efficient expression of a Tislelizumab antibody or antigen-binding fragment. These elements typically include an ATG initiation codon and adjacent ribosome binding site or other sequences. In addition, the efficiency of expression can be enhanced by the inclusion of enhancers appropriate to the cell system in use (see, e.g., Scharf et al., Results Probl. Cell Differ. 20:125, 1994; and Bittner et al., Meth. Enzymol., 153:516, 1987). For example, the SV40 enhancer or CMV enhancer can be used to increase expression in mammalian host cells.

The host cells for harboring and expressing the Tislelizumab antibody chains can be either prokaryotic or eukaryotic. E. coli is one prokaryotic host useful for cloning and expressing the polynucleotides of the present disclosure. Other microbial hosts suitable for use include bacilli, such as Bacillus subtilis, and other enterobacteriaceae, such as Salmonella, Serratia, and various Pseudomonas species. In these prokaryotic hosts, one can also make expression vectors, which typically contain expression control sequences compatible with the host cell (e.g., an origin of replication). In addition, any number of a variety of well-known promoters will be present, such as the lactose promoter system, a tryptophan (trp) promoter system, a beta-lactamase promoter system, or a promoter system from phage lambda. The promoters typically control expression, optionally with an operator sequence, and have ribosome binding site sequences and the like, for initiating and completing transcription and translation. Other microbes, such as yeast, can also be employed to express Tislelizumab. Insect cells in combination with baculovirus vectors can also be used.

In other aspects, mammalian host cells are used to express and produce Tislelizumab. For example, they can be either a hybridoma cell line expressing endogenous immunoglobulin genes or a mammalian cell line harboring an exogenous expression vector. These include any normal mortal or normal or abnormal immortal animal or human cell. For example, a number of suitable host cell lines capable of secreting intact immunoglobulins have been developed, including the CHO cell lines, various COS cell lines, HEK 293 cells, myeloma cell lines, transformed B-cells and hybridomas. The use of mammalian tissue cell culture to express polypeptides is discussed generally in, e.g., Winnacker, From Genes to Clones, VCH Publishers, NY, N.Y., 1987. Expression vectors for mammalian host cells can include expression control sequences, such as an origin of replication, a promoter, and an enhancer (see, e.g., Queen et al., Immunol. Rev. 89:49-68, 1986), and necessary processing information sites, such as ribosome binding sites, RNA splice sites, polyadenylation sites, and transcriptional terminator sequences. These expression vectors usually contain promoters derived from mammalian genes or from mammalian viruses. Suitable promoters can be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPSV promoter, the tetracycline-inducible CMV promoter (such as the human immediate-early CMV promoter), the constitutive CMV promoter, and promoter-enhancer combinations known in the art.

### EXAMPLES

The examples and description of certain embodiments should be taken as illustrating, rather than as limiting the present disclosure as defined by the claims. As will be readily appreciated, numerous variations and combinations of the features set forth above can be utilized without departing from the present disclosure and as set forth in the claims. All such variations are intended to be included within the scope of the present disclosure. All references cited are incorporated herein by reference in their entireties.

### Analytical Methods

This methods section provides a summary of the methods used in the following Examples 1-5.

### SEC-HPLC

Formation of soluble aggregates is analyzed by size exclusion chromatography (SEC) on a Waters HPLC system. Protein is separated based on molecular size on a TSKgel G3000™ SWXL column maintained at 37±5° C. using an isocratic gradient. Molecular weight species are eluted and detected by IN absorption at 280 nm. The distribution of aggregates, monomer and fragments are quantitated via the peak areas for standards and samples.

### IEC

The charge heterogeneity of a sample is determined by using Alliance™ HPLC System™ (Waters) with an ion exchange chromatography method (IEC). Based on the interactions between charges, the separation takes advantage of small differences in electric charge in charged molecules. The samples are analyzed in their native state, when using ahigh conductivity elution buffer, a specific peak pattern will show the various charge variants of the antibody (acidic, basic and main charge variants). Samples are injected by pressure and the mobilized proteins are detected by UV absorbance at 280 nm.

### CE-SDS(NR)

The purity of sample is determined using PA800 Plus™ (Beckman) by a capillary gel electrophoresis (CE) method. Samples are denatured with sodium dodecyl sulphate (SDS) and separated based on size in a capillary filled with a gel that acts as a sieving medium. In non-reduced (NR) samples, an alkylating agent, N-Ethylmaleimide (NEM), is added to avoid any fragmentation induced by sample preparation and to ensure that the main IgG peak remains intact. Samples are injected electrokinetically and the mobilized proteins are detected by UV absorbance at 200 nm using a UV detector. The reportable value for non-reduced samples is the time corrected area percent (TCA) % of the IgG main peak.

### Protein Concentration

Protein concentrations are determined at UV 280 nm.

### Viscosity

The viscosity of the antibody formulations is measured on a chip-based microVISC™ instrument (Rheosense), in which the pressure difference correlates with solution dynamic viscosity. Sample size is approximately 70-100 μL. Aliquots are loaded into a 400 μL microVISC™ disposable pipette and connected to the chip. Triplicate measurements are taken at a shear rate of 500 S⁻¹ and at a temperature of about 25° C.

### Osmolality

The osmolality of the antibody solution or buffer solution is measured by OSMOMAT 3000™ osmolality tester (Gonotec). 50 μl of each sample is loaded twice and tested to obtain the average osmolality value.

### Example 1: Define Parameters of the SPTFF Process in the UF1 Step

In order to define the parameters of the SPTFF for a high concentration PD1 antibody solution, a lab scale UF/DF system and process were designed to implement scale up and future large scale CMP production. The unit operation contained several steps: membrane pre-use treatment (WFI flush, Integrity test, CIP, NWP test), equilibrium, ultrafiltration 1 (SPTFF), diafiltration, ultrafiltration 2, system flush and recovery, UF/DF pool, membrane post-use treatment etc., which are shown in the process flowchart (FIG. 1A). The UF/DF system was comprised of three 0.11 m² Pellion3 Ultracel™ 30 kDa, D membrane in SPTFF skid 1 and a conventional UF/DF 0.11 m² Pellion3 Ultracel™ 30 kDa, D membrane with membrane housing skid 2, shown in the diagram in FIG. 1B. The fluid paths in this system were designed to minimize the system dead volume in order to reduce the dilution effect by the system flush.

To determine the process parameters for SPTFF process (UF1), Tislelizumab was prepared and purified after a viral filtration step as the UF/DF process feed solution. The antibody was dispersed in a process feed solution of 50 mM acetate, pH 5.36 buffer with an antibody concentration of 8 and 20 g/L and filtered by 0.2 μm Corning™ filtration system. To evaluate the relationship between post-membrane pressure and flux, feed concentration and VCF, a lab scale UF/DF system following the design in FIG. 1B was assembled for testing. The flux was controlled at 0.05-0.4 L/min/m², and post-membrane pressure at 10, 15 and 20 Psi for SPTFF.

FIG. 1C demonstrates the VCF changes with flux, initial concentration and post-membrane pressure. The VCF value increases with flux decreasing. In high concentration group, 20 g/L initial concentration solution achieves 4-6 times concentration factor at low flux 0.1 L/min/m², up to about 100 g/L. When flux increases to 0.4 L/min/m², the VCF value decreases to about 2 times. In 8 g/L low concentration group, the same trend exists but higher VCF (8-10 times) can be achieved at low flux 0.1-0.2 L/min/m² due to low initial feed concentration. In both experimental groups, post-membrane pressure has no significant impact on VCF in 10-20 Psi range.

### Example 2: SPTFF Process as the UF1 Step for Different Feed Concentration

Tislelizumab was prepared and purified after a viral filtration step as the UF/DF process feed solution. The antibody was dispersed in a process feed solution of 50 mM acetate, pH 5.36 buffer with an antibody concentration of 8, 14 and 20 g/L and filtered by 0.2 μm Corning™ filtration system. The same lab scale UF/DF system in Example 1 was used for testing in this example. The post-membrane pressure is controlled at about 15 Psi and feed flux from 0.05-0.4 L/min/m² in UF1 SPTFF step.

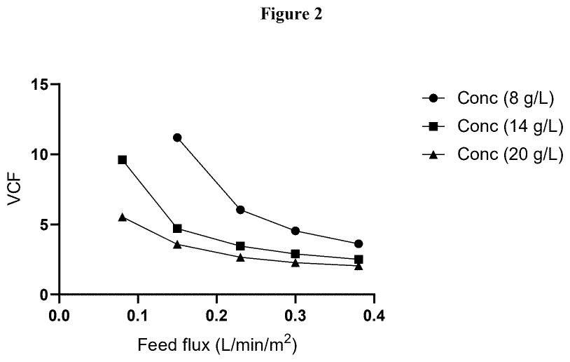

FIG. 2 shows the VCF decreases with flux increasing. Low initial concentration has higher VCF than higher initial concentration at same flux condition. The difference of VCF values for different initial feed concentration at higher flux is smaller than that at lower flux. In order to obtain the UF1 pool after SPTFF with concentration about 30-70 g/L, flux in 0.15-0.4 L/min/m² range is suitable for all initial feed concentration in this case.

### Example 3: SPTFF Process as the UF1 Step at Increased Temperature

The same Tislelizumab solution and lab skid as that in Example 1 were prepared and used in this example. The feed concentration was adjusted to 8 and 14 g/L. The post-membrane pressure of the SPTFF for UF1 was controlled at 15 Psi and temperature controlled at room temperature (RT) and 35° C.

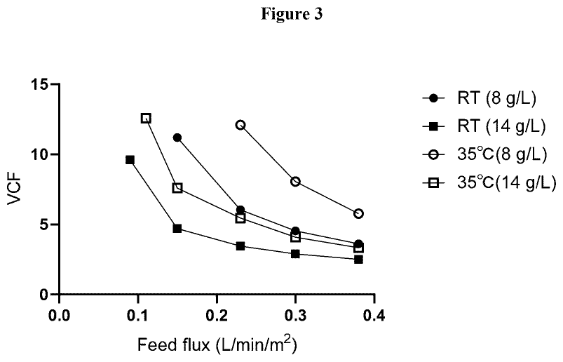

FIG. 3 shows that a higher operational temperature (35° C.) condition achieves a higher VCF value than RT at any flux conditions and any initial feed concentrations. High temperature will decrease solution viscosity and thus increase the flux through membrane. At 8 g/L, increasing temperature to 35° C. is able to achieve VCF 2 times that of RT. The VCF increasing effect caused by temperature reduces with solution concentration increasing and flux increasing. However, this observation means the SPTFF process can be operated at 35° C. under higher flux, while keeping same VCF, and shorten total process time if needed.

### Example 4: UF/DF Unit Operation with SPTFF as the UF1 Step for SubQ Manufacturing

The same Tislelizumab solution and lab skid as that in Example 1 were prepared and used in this example. The feed concentration was adjusted to 8.23 g/L. The membrane loading capacity was 265.92 g/m² in SPTFF UF1 and 797.76 g/m² in DF and UF2. The unit operation contained membrane pre-use treatment (WFI flush, Integrity test, CIP, NWP test), equilibrium, ultrafiltration 1 SPTFF, diafiltration, ultrafiltration 2, system flush and recovery, UF/DF pool, membrane post-use treatment etc., are shown in the process flowchart (FIG. 1A).

The post-membrane pressure was controlled at about 15 Psi and feed flux at ˜0.3 L/min/m² in UF1 SPTFF step. The protein concentration was concentrated to 40 g/L in UF1 pool. Then, the DF and UF2 process were carried out in conventional UF/DF skid 2. After 6 exchange volume of DF buffer (20 mM His-His HCl, 70 mM NaCl with pH 6.04), the DF pool solution has protein concentration at 39.4 g/L with pH at 6.1. The DF pool solution was further processed in UF2 step with TMP controlled at about 14.5 Psi (with an upper limit of 29 Psi) and flux at 5 L/min/m². The over concentrated pool achieved 221.08 g/L antibody protein concentration. After flushing and recycling the whole UF/DF system with a volume of DF buffer, the final UF2 pool had an antibody protein concentration at 186.67 g/L, in 20 mM His-His HCl, 70 mM NaCl, pH 6.0 buffer.

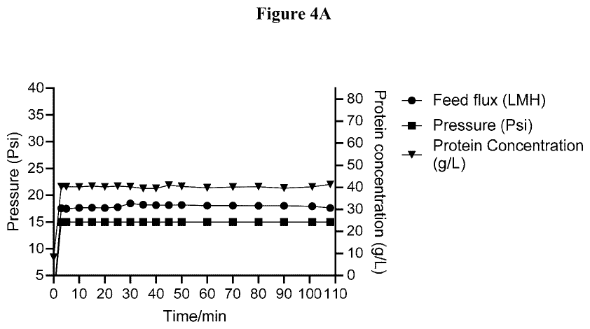

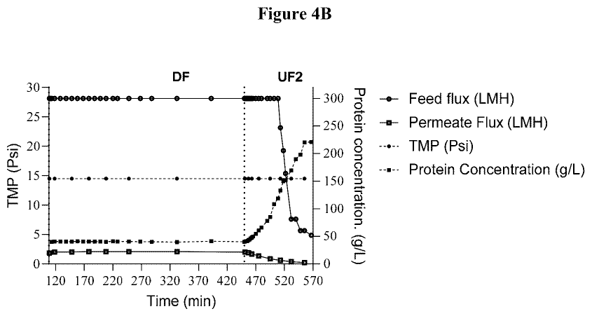

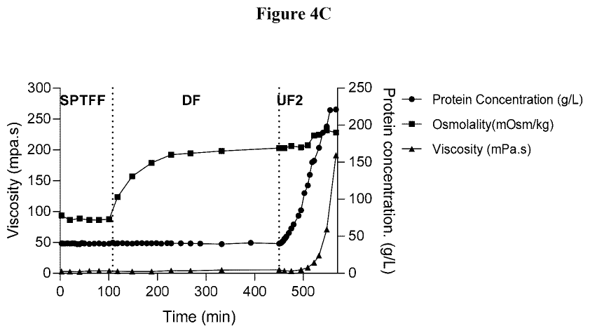

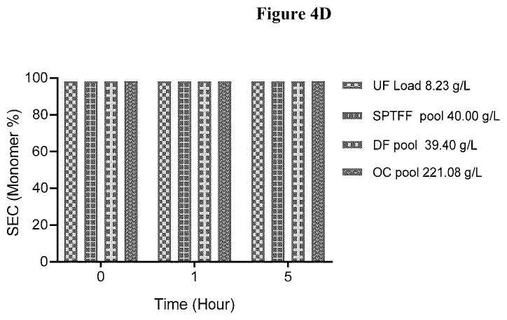

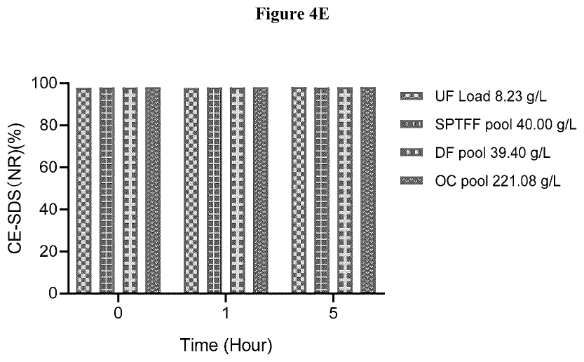

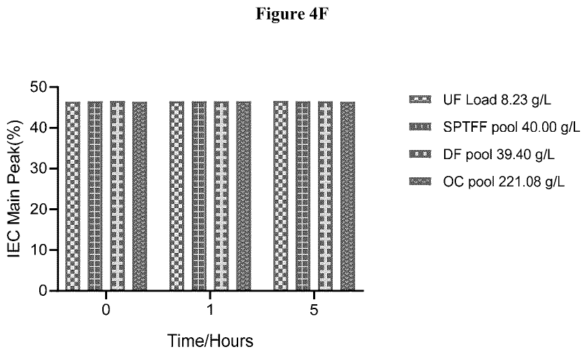

FIG. 4A shows the process chart of UF1 SPTFF step. Protein concentration increased to about 40 g/L immediately due to the mechanism of SPTFF. It took about 110 minutes to process all required solution volume to achieve the UF1 pool. FIG. 4B shows the process chart of DF/UF2 step in conventional UF/DF process by keeping TMP at 14.5 Psi with final concentration achieving 221 g/L. FIG. 4C shows the osmolality and viscosity curves with antibody protein concentration changing in SPTFF UF1/DF/UF2 steps. Osmolality and viscosity increased exponentially when antibody protein concentration was over 100 g/L in the UF2 step. The quality data (SEC, CE-SDS (NR) and IEC) shown from FIG. 4D to FIG. 4F demonstrated that the over concentrated pool and intermediate solutions of each step (SPTFF pool and DF pool) were quality consistent through this process operation and stable up to 5 hours, indicating that this process maintained the integrity of the Tislelizumab antibody.

### Example 5: UF/DF Unit Operation with SPTFF as the UF1 Step for SubQ Manufacturing with Increased Temperature

It is known that solution viscosity decreases with increasing solution temperature. Theoretically, operating UF/DF at higher temperate, for example, 35° C., will show better process performance, for example, more even TMP and better flux control, or be able to achieve a higher concentration than the process at room temperature or lower temperature. To evaluate the temperature effects on this UF/DF process, the same Tislelizumab solution and lab skid as that in Example 1 were prepared and used in this example. The feed concentration was adjusted to 8.27 g/L. The membrane loading capacity was 268.71 g/m² in SPTFF UF1 and 806.13 g/m² in DF and UF2. The unit operation contained membrane pre-use treatment (WFI flush, Integrity test, CIP, NWP test), equilibrium, ultrafiltration 1 SPTFF, diafiltration, ultrafiltration 2, system flush and recovery, UF/DF pool, membrane post-use treatment etc., are shown in the process flowchart (FIG. 1A).

The post-membrane pressure was controlled at about 15 Psi and feed flux at ˜0.3 L/min/m² in UF1 SPTFF step. The protein concentration was concentrated to 68.21 g/L in the UF1 pool. Then, the DF and UF2 process were carried out in a conventional UF/DF skid 2. After 6 exchange volumes of DF buffer (20 mM His-His HCl, 70 mM NaCl with pH 6.04), the DF pool solution has a protein concentration at 66.25 g/L with pH at 5.98. The DF pool solution was further processed in the UF2 step with TMP controlled at about 14.5 Psi (with an upper limit of 29 Psi) and flux at 5 L/min/m². The over concentrated pool achieved a 224.73 g/L antibody protein concentration. After flushing and recycling the whole UF/DF system with a volume of DF buffer, the final UF2 pool had an antibody protein concentration at 187.31 g/L, in 20 mM His-His HCl, 70 mM NaCl, pH 6.0 buffer.

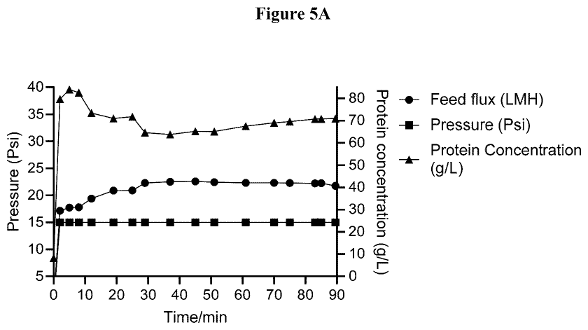

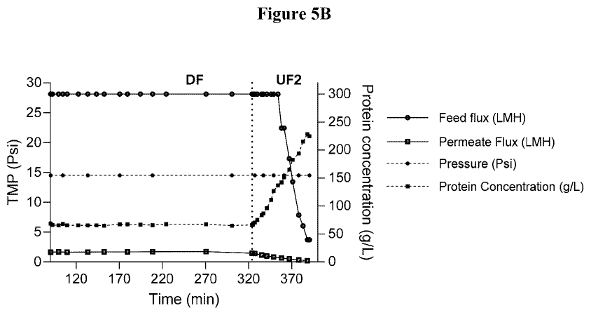

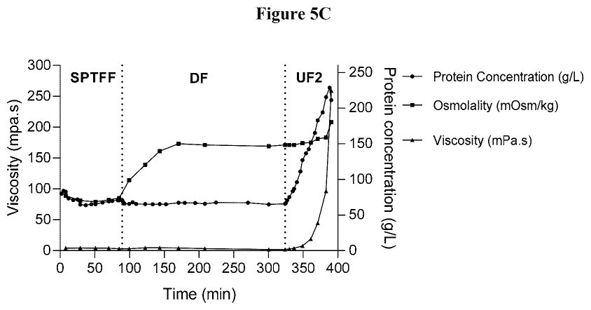

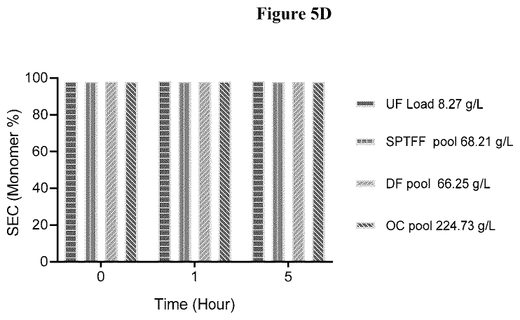

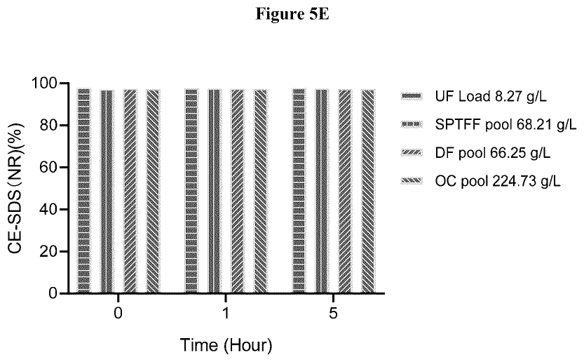

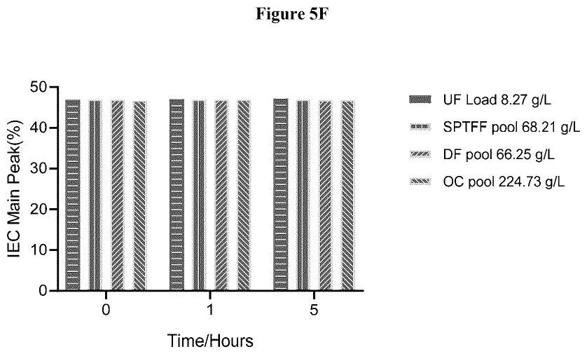

FIG. 5A shows the process chart of UF1 SPTFF step. Protein concentration increased to ˜70 g/L immediately due to the mechanism of SPTFF. It took about 90 minutes to process all required solution volume to achieve UF1 pool. The UF1 process time was shortened due to higher feed and permeate flux at higher operational temperature. FIG. 5B shows the process chart of the DF/UF2 step in a conventional UF/DF process by keeping TMP at 14.5 Psi with final concentration achieving 224.73 g/L. FIG. 5C shows the osmolality and viscosity curves with antibody protein concentration changing in the SPTFF UF1/DF/UF2 steps. Osmolality and viscosity increased exponentially when antibody protein concentration was beyond 100 g/L in the UF2 step. The total processing time at 35° C. was about 390 minutes (FIG. 5B), which was about 177 minutes faster than the total processing time at RT (FIG. 4B) due to higher temperature effect on solution viscosity and permeate flux. The quality data (SEC, CE-SDS (NR) and IEC) shown from FIG. 5D to FIG. 5F demonstrated even the over concentrated pool and intermediate solutions of each step (SPTFF pool and DF pool) were quality consistent through this process operation and stable up to 5 hours, indicating that this high temperature process maintained the integrity of the Tislelizumab antibody.

1. An ultrafiltration (UF)/diafiltration (DF) process for a highly concentrated solution comprising a PD1 antibody or antigen binding fragment thereof, the process comprising the steps of:
(a) ultrafiltrating the PD1 antibody or antigen binding fragment thereof in a process feed material to obtain an ultrafiltration 1 (UF1) pool protein with an intermediate antibody concentration through a single-pass tangential flow filtration (SPTFF) operation;
(b) diafiltrating the UF1 pool protein from step (a) with diafiltration (DF) buffer into a final drug substance formulation buffer, to obtain a DF pool;
(c) ultrafiltrating the DF pool from step (b) into a high concentration solution as an over concentrated pool with a desired concentration; and
(d) adjusting the over concentrated pool to a final drug substance target concentration to prepare a UF2 pool, and then further diluting the UF2 pool to the highly concentrated solution.

2. The process of claim 1, wherein the PD1 antibody or antigen binding fragment thereof, comprises:
(a) a HCDR (Heavy Chain Complementarity Determining Region) 1 of SEQ ID NO: 1,
(b) a HCDR2 of SEQ ID NO:2, and
(c) a HCDR3 of SEQ ID NO:3 and a light chain variable region that comprises:
(i) a LCDR (Light Chain Complementarity Determining Region) 1 of SEQ ID NO:4,
(ii) a LCDR2 of SEQ ID NO:5, and
(iii) a LCDR3 of SEQ ID NO:6.

3. The process of claim 1, wherein the PD1 antibody or antigen binding fragment thereof, comprises SEQ ID NO:7 and SEQ ID NO:8.

4. The process of claim 1, wherein the process feed material in step (a) comprises a buffer, and wherein the buffer is selected from the group consisting of histidine, acetate, citrate, succinate, phosphate, a mixture of histidine and acetic acid, and a mixture of histidine and citric acid.

5. The process of claim 4, wherein the process feed material comprises a buffer, and wherein the buffer comprises histidine, a mixture of histidine and acetic acid, or a mixture of histidine and citric acid.

6. The process of claim 1, wherein the highly concentrated solution is at a concentration of about 8 g/L to about 20 g/L.

7. The process of claim 1, wherein the steps (a)-(c) comprise a 30 kDa SPTFF membrane and a 30 kDa UF/DF membrane.

8. The process of claim 7, wherein the membrane loading capacity is about 35-about 270 g/m² for the SPTFF membrane and about 100-about 800 g/m² for the UF/DF membrane.

9.-11. (canceled)

12. The process of claim 1, wherein in step A the UF1 pool protein concentration is a range of about 30-about 70 g/L.

13. The process of claim 1, wherein step A results in a volume concentration factor (VCF) between about 2-about 10.

14.-16. (canceled)

17. The process of claim 1, wherein in step (b) the UF1 pool protein has a protein concentration between about 30-about 70 g/L.

18. The process of claim 17, wherein the UF1 pool protein has a concentration of about 50 g/L.

19.-25. (canceled)

26. The process of claim 1, wherein in step C the DF pool has a protein concentration between about 30-about 70 g/L.

27. The process of claim 26, wherein the protein concentration is about 50 g/L.

28. The process of claim 1, wherein in step D the over concentrated pool has a protein concentration of about 100 g/L to about 225 g/L.

29. The process of claim 1, wherein the UF2 pool is prepared by diluting the over concentrated pool in a different dilution ratio, and has concentration up to about 225 g/L.

30. The process of claim 29, wherein the UF2 pool is prepared by diluting the overconcentrated pool to about 165 g/L.

31. The process of claim 21, wherein the UF2 pool in step D is buffered with histidine.

32. The process of claim 31, wherein the concentration of histidine is about 15 mM to about 25 mM.

33. The process of claim 32, wherein the buffer comprises 20 mM histidine buffer with pH between about 5.5-about 6.0.

34.-36. (canceled)
